(19) United States
(12) Patent Application Publication
(10) Pub. No.: US 2024/0188828 A1
(43) Pub. Date: Jun. 13, 2024

(54) EASY INSERT FINGER SENSOR FOR TRANSMISSION BASED SPECTROSCOPY SENSOR

(71) Applicant: Cercacor Laboratories, Inc., Irvine, CA (US)

(72) Inventor: Gregory A. Olsen, Lake Forest, CA (US)

(21) Appl. No.: 18/544,108

(22) Filed: Dec. 18, 2023

Related U.S. Application Data
(62) Division of application No. 16/393,158, filed on Apr. 24, 2019, now Pat. No. 11,883,129.
(60) Provisional application No. 62/662,142, filed on Apr. 24, 2018.
(60) Provisional application No. 62/680,170, filed on Jun. 4, 2018.

(51) Int. Cl. A61B 5/00

(52) U.S. Cl. CPC A61B 5/0075; A61B 5/6826; A61B 5/6838; A61B 2560/0425; A61B 2560/0443

(57) ABSTRACT

An optical physiological finger sensor system including an ergonomic interface shaped into a natural curve of a user's hand and finger

### INCORPORATION BY REFERENCE TO ANY PRIORITY APPLICATIONS

This application is a continuation of U.S. application Ser. No. 16/393,158, filed Apr. 24, 2019, which claims the priority benefit of U.S. Application No. 62/662,142, filed Apr. 24, 2018, and U.S. Application No. 62/680,170, filed Jun. 4, 2018, each of which are hereby incorporated by reference in its entirety herein.

Any and all applications for which a foreign or domestic priority claim is identified in the Application Data Sheet as filed with the present application are hereby incorporated by reference under 37 C.F.R. § 1.57.

### BACKGROUND

### Field of Use

The present disclosure relates generally to the field of patient monitoring devices.

### Description of Related Art

Physiological monitors measure many important parameters useful in providing care to a patient. For example, one such physiological monitor is a pulse oximeter. Many physiological monitoring devices that exist on the market utilize a reusable alligator clip type sensor. These kinds of sensors are usually placed on a finger to measure noninvasive physiological parameters and biomarkers through transmission spectroscopy. A user's finger is placed into an alligator clip sensor and one or more light emitters emit light into the tissue of the patient and one or more light detectors detect the attenuated light after transmission through or reflection from the tissue.

### SUMMARY

The present disclosure provides a physiological monitoring device. Examples of the present disclosure relate to systems that allow an easy insert finger sensor. In particular, but without limitation, embodiments disclosed herein relate to spectroscopy technologies.

A physiological finger sensor system may include an ergonomic interface, where the ergonomic interface is shaped into a natural curve of a user's hand and a finger.

The finger sensor further can include a pivot release stand. The system can include a pin and the pin can include a coil spring. The pivot release stand can be located at the deepest end of the first and second soft pads. The ergonomic interface can include a display. The system can include an algorithm board. The system can include a processor. The system can include a communication board. The system can include a battery. The algorithm board can include RainbowSET® spectroscopy algorithms that can measure at least nine parameters from transmission based spectroscopy. The interface can be a rounded shape.

The finger sensor can be at a maximum opening when a finger is not inserted. The finger sensor can be at a closed position when a finger is not inserted. The finger sensor can be closed prior to a finger insertion. The finger sensor can close once a finger is fully inserted and when the finger contacts the pivot release stand. The pivot release stand can be on a spring system that can be able to return to an original position when a user's finger is removed out of the sensor and thus leaving the sensor in the most open position while waiting for the next finger insertion.

The LED emitter can transmit at least a signal through a finger to the detector. The processor can calculate data based upon signals collected by the detector.

The finger sensor can be a kickstand sensor. The finger sensor can be a bi-directional kickstand sensor. The finger sensor can be a scissor over sensor. The finger sensor can include: a top portion, where the top portion can have a first soft pad and at least one light emitter; and a bottom portion, where the bottom portion can have a second soft pad and at least one detector. The finger sensor can include a bottom portion, wherein the bottom portion can have a first soft pad and at least one light emitter; and a top portion, where the top portion can have a second soft pad and at least one detector. The finger sensor can be a reflectance-based sensor. The finger sensor can be a transmission-based sensor and a reflectance-based sensor.

A finger sensor system can include: a first housing component that can include a first sensor and a first finger placement component configured to support the first sensor and secure a first finger near the first sensor; a second housing component that can include a second sensor and a second finger placement component configured to support the second sensor and secure a second finger near the second sensor; and a display component.

The first housing component can be disposed on the second housing component.

The first sensor can be a spectroscopic sensor configured to measure Raman emissions. The second sensor can be an optical sensor configured to measure pulse oximetry.

The first finger placement component can include a sensor cover and an internal clasp. The internal clasp can include a hinged component configured to support the first finger. The sensor cover can include the first sensor. The internal clasp can include a spring configured to push the hinged component towards the sensor cover.

The first finger placement component can include a nail lock configured to secure a fingernail of the first finger. The nail lock can be configured to mate with a nail guide. The nail guide can be secured onto the fingernail. The nail guide can be secured using an adhesive.

The display component can be configured to display data associated with at least one physiological parameter. The at least one physiological parameter can include glucose. The data can include the physiological parameter. The data can include a graphical representation of variation in the physiological parameter over time.

The third housing component can be configured to support at least one power source for the first sensor and the second sensor.

A finger sensor system can include: a first housing component that can include a first sensor and a first finger placement component configured to support the first sensor and secure a first finger near the first sensor; and a display component.

The finger sensor system can include a second housing component comprising a second sensor and a second finger placement component configured to support the second sensor and secure a second finger near the second sensor.

The finger sensor system can include a third housing component comprising one or more hardware processors.

A finger sensor system can include: one or more modular components that can include at least one finger well and at least one sensor adjacent to the at least one finger well.

The finger well can be configured to be embedded in a central portion of the one or more modular components. The finger well can be configured to be adjacent to a central portion of the one or more modular components.

The finger well can include at least one pressure component. The at least one pressure component can include a spring configured to push the at least one sensor towards an interior of the finger well. The at least one pressure component can include a clasp configured to apply pressure to a measurement site of a patient.

The finger sensor system can include an alignment lens configured to be disposed between a finger disposed in the finger well and the at least one sensor. The alignment lens can be a flexible material. The alignment lens can be silicone.

### BRIEF DESCRIPTION OF THE DRAWINGS

FIG. 1 illustrates a top view of an example device.

FIG. 2 shows a cross-section of the device of FIG. 1.

FIG. 3 shows another detailed cross section of the device of FIG. 1.

FIGS. 4A and 4B show how a pivot kickstand sensor can be used on a device.

FIGS. 5A and 5B show how a bi-directional kickstand sensor can be used on a device.

FIGS. 6A and 6B show how a scissor over sensor can be used on a device.

FIGS. 7A-7E illustrate perspective views of an example multi-sensor device.

FIG. 8A illustrates an example multi-sensor device with separable sensor units.

FIG. 8B illustrates an exploded view of the multi-sensor device of FIG. 8A.

FIGS. 9 and 10 shows cross-sectional views of an example multi-sensor device.

FIGS. 11A and 11B show different cross-sectional views of an example device with finger placement component for a Raman sensor

FIG. 12A shows an example nail guide for use with an example device.

FIG. 12B shows an example nail guide lock for use with an example device.

FIGS. 13A and 13B illustrate example display modes of an example device.

FIGS. 14A-14D illustrate perspective views of an example device that allows for a tissue site to sit inside a center housing portion of the device.

FIGS. 15A and 15B illustrate perspective views of an example device that allows for a tissue site to sit adjacent to a center housing portion of the device.

FIGS. 16A-16C show example sensor pressure components of the device.

### DETAILED DESCRIPTION

### Overview

Examples disclosed herein relate to systems that allow an easy insert finger physiological sensor. These systems can be used on transmission-based spectroscopy technologies or reflectance-based spectroscopy technologies. Currently, many devices on the market place an alligator clip type sensor on a finger to measure parameters and biomarkers noninvasively. Additional challenges exist with current pulse oximetry and co-oximetry noninvasive sensors. Current pulse oximetry and co-oximetry noninvasive sensors require a user to place his or her finger in a clothespin style clip. This action can require both hands of the patient or a clinician to ensure accurate placement. Additionally, placement accuracy of the emitter and detector windows relative to the patient's measurement site can be difficult to achieve with an alligator clip type sensor. Placement of the windows is important in obtaining a value when measuring. Systems and methods described herein seek to improve the placement of transmission and reflectance based spectroscopic sensors at a patient tissue site. For example, in the case of a finger, a device that allows for consistent and ergonomic finger placement with relation to the sensor can allow for more consistent sensor measurements due to ease of use and increased precision of tissue site placement. Systems and methods described herein relate to an object that the hand is placed onto, around or within. The present disclosure provides an inviting ergonomic experience for a user.

### Components of an Example Sensor Device

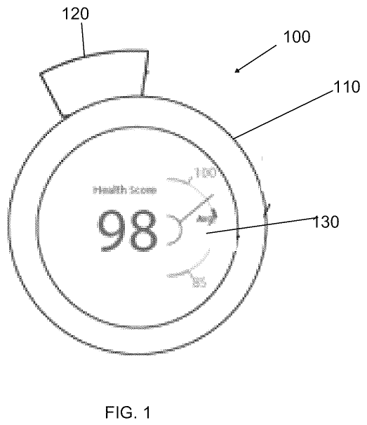

FIG. 1 shows a device 100. The device 100 can have a rounded form structured shape 110 with a natural curve so as to be inviting for a user's hand. The device 100 can embody all required hardware to measure one or multiple parameters directly from reflectance-based or transmission-based spectroscopy. The device 100 can have a finger sensor component 120 and a display 130. The device 100 can have a structure in the back such as a ring or finger/hand anchor to prevent the user from dropping the device 100. The device 100 can have a physical port, such as a USB port, to connect with smart phones, computers, tablets, or other devices to transmit data. The device 100 can also wirelessly transmit data. The device can have a processor to further process collected data. The device 100 can have a rechargeable battery port, USB charging, wireless charging components (such as but not limited to Qi), or a direct power input port. The device 100 can have audio features such as a speaker, microphone, audio output, and volume adjustment. The device 100 can have display brightness or contrast features. The display 130 can be a touchscreen. The display 130 can also be an integrated button. The display 130 can have capacitance or projected capacitance abilities to respond to touch inputs. The device 100 can have a screen lock feature. The device 100 can be made out of drop resistant material. The display can be made out of scratch resistant or shatter resistant material. The device 100 can come in different sizes for adult or child use. The display 130 can flash different colors to indicate a status of the user.

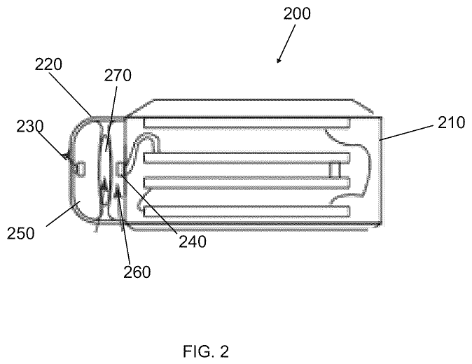

FIG. 2 shows a cross-section of the device 200. A cross section of the form structured shape 210 and a cross section of the finger sensor 220 are presented. The finger sensor 220 can be a modular attachment to the device 200. The finger sensor 220 can have at least one emitter 230 and at least one detector 240. The emitter 230 can be an LED. The detector 240 can be in the inner most location of the finger sensor. The finger sensor 220 can have two soft pads 250, 260 for the user's finger when the finger is inserted.

The two halves of the finger sensor can be connected with a pivot-release stand. The pivot release stand can have a pin and coil spring that places a specific amount of force on to the finger being measured. The pivot release stand can be placed at the deepest end of the sensor's soft pads 250, 260. A momentum-based spring can apply pressure from either the top or bottom sensor pads. A momentum-based spring can also be used in a mechanical reaction in response to a user inserting his or her finger. An electronic trigger can also be used in response to a user inserting his or her finger into the sensor portion. The pivot release stand can be in an open position prior to inserting a finger. The pivot release stand can be in a closed position prior to inserting a finger. The pivot release stand can be opened and closed by a lever, switch, or button. When the finger is removed, the pivot release stand can return to its original position and can open the sensor.

A spring kickstand 270 can be placed in the finger sensor 220. Prior to inserting a finger, the spring kickstand 270 of the finger sensor 220 can be at a maximum opening. The spring kickstand 270 of the finger sensor 220 can also be in a closed position prior to finger insertion. The finger sensor can also be in connection with a lever, switch, or button to open the finger sensor 220 prior to finger insertion. When a finger is inserted and pushes or contacts the spring kickstand 270, the spring kickstand can become under tension and close the finger sensor 220. Optionally, a user can use a different finger to manipulate the spring kickstand 270 to cause the finger sensor 220 to open and close. A switch, button, or lever can be located externally and be in connection with the spring kickstand 270. The finger sensor can also have dual entry points for two fingers or an opposite entry point for another finger. The finger sensor can also be located on the back of the device 200 or on the front of the device 200.

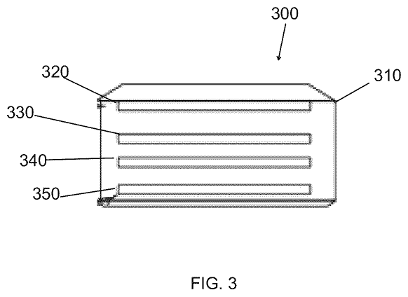

FIG. 3 shows a detailed example of a cross section of the form structured shape 310 or the device 300. The device 300 can contain a display 320. The device 300 can contain an algorithm board 330. The device 300 can contain a communication board 340. The device 300 can have a battery 350. The algorithm board 330 can use algorithms such as the RainbowSETX, available from Masimo corporation of Irvine, CA, or other algorithms to process the data collected by the detector. The battery 350 can be a lithium battery. The battery 350 can be rechargeable. The battery 350 can have wireless or wire based charging. The components of the device 300 described herein can be modular.

### Operation of Example Sensor Device

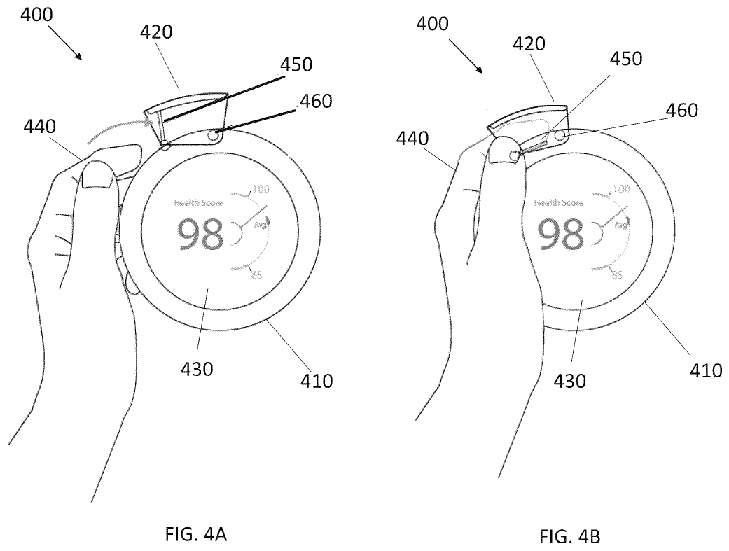

FIGS. 4A and 4B shows a pivot kickstand sensor 420 that can be used on a device 400. FIG. 4A and FIG. 4B shows a user can use his or her finger 440 to use the device 400. As shown in FIG. 4A, the pivot kickstand sensor 420 can be opened to a maximum size prior to finger insertion. The pivot kickstand sensor 420 has a kickstand 450 that can be upright prior to finger insertion. The pivot kickstand sensor top can have a spring-loaded rotational pivot 460.

As shown in FIG. 4B, the pivot kickstand 420 can close once the finger is inserted and hits the kickstand 450 or pivot release stand. The user can hold the device 400 based on the contours of the form structured shape 410. When a user's finger 440 enters the pivot kickstand sensor 420, the user can feel the walls of the pivot-release stand press down. This movement against the pivot release stand wall can release the downward clamping force of the pivot kickstand sensor 420. This pivot release stand can also be on a spring system. The pivot kickstand sensor 420 can be closed once the finger 440 is inserted and hits the kickstand 450. When a user's finger 440 is removed from the pivot kickstand sensor 420, the kickstand 450 can engage back into place and can leave the kickstand pivot kickstand sensor 420 in an open position while waiting for the next finger insertion. The display 430 can show the data that the detector collects from the emitters, instructions for the user, commands to the user, or other indications as described herein.

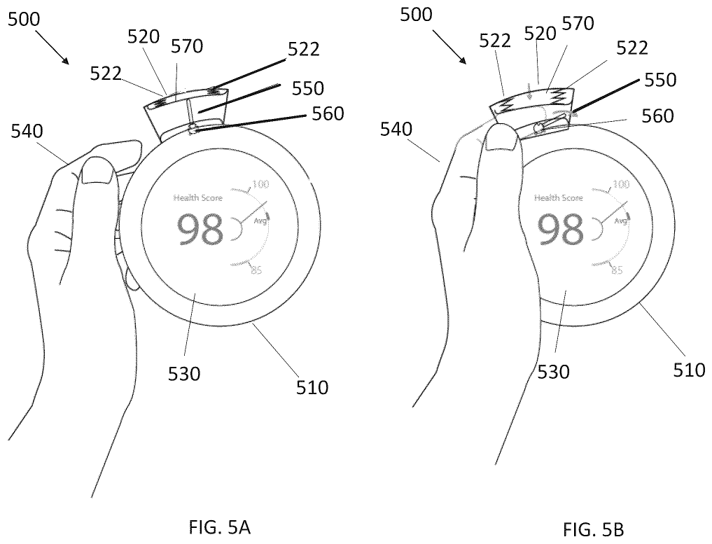

FIGS. 5A and 5B shows a bi-directional kickstand sensor 520 can be used on a device 500. FIG. 5A and FIG. 5B shows a user can use his or her finger 540 to use the device 500. The bi-directional kickstand sensor 520 allows either hand to operate the device 500. As shown in FIG. 5A, momentum springs 522 can be in a loaded position prior to finger 540 insertion. The finger kickstand 550 can also be in an upright position prior to finger insertion. A pivot with a rotational spring 560 can also be used. As shown in FIG. 5A, the bi-directional kickstand sensor 520 can be opened to a maximum size prior to finger insertion. The user places his or her finger 540 into the kickstand bi-directional kickstand sensor 520 sheath until a clicking sound or other audible signal can be made. The sound or audible signal can notify the user of proper finger 540 placement. A pivot notch can create the clicking sound. Alternatively, the device's pivot notch can also provide a tactile feedback system to signal to the user that the finger is placed properly.

As shown in FIG. 5B, once the finger is inserted and hits the finger kickstand 550 or pivot release stand, the bi-directional kickstand sensor 520 can close. The user can hold the device 500 based on the contours of the form structured shape 510. When a user's finger 540 enters the bi-directional kickstand sensor 520, the finger kickstand can be held down by the finger 540. The user can also feel the top pad 570 extend to press against the finger 540. When a user's finger 540 is removed from the bi-directional kickstand sensor 520, the finger kickstand 550 can engage back into place and can leave the sensor in an open position to wait for the next finger insertion. The display 530 can show the data that the detector collects from the emitters, instructions for the user, commands to the user, or other indications as described herein.

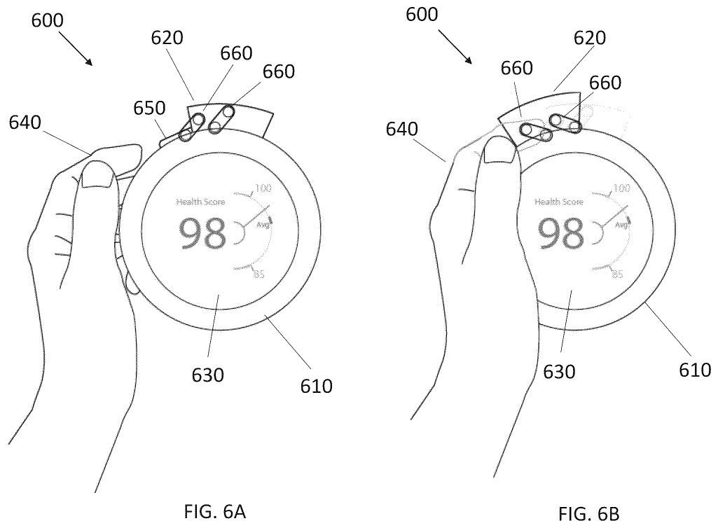

FIGS. 6A and 6B shows a scissor over sensor 620 can be used on a device 600. FIG. 6A and FIG. 6B shows a user can use his or her finger 640 to use the device 600. As shown in FIG. 6A, the scissor over sensor 620 has a bottom pad 650. Prior to a finger 640 insertion, the scissor over sensor 620 can be in an open position. When a user places his or her finger 640 on the bottom pad 650, the pivots 660 of the scissor over sensor 620 activate to slide the scissor over sensor 620 over the finger 640. The user can hold the device 600 based on the contours of the form structured shape 610.

As shown in FIG. 6B, the scissor over sensor 620 can close while the user is pressing on the bottom pad 650. When the user removes the finger 640, the scissor over sensor 620 can return to the original open position. The display 630 can show the data that the detector collects from the emitters, instructions for the user, commands to the user, or other indications as described herein.

### Example Multi-Sensor Device

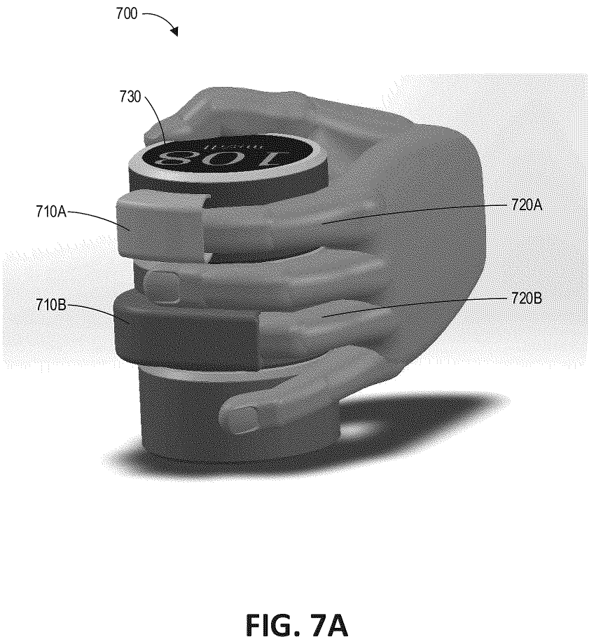

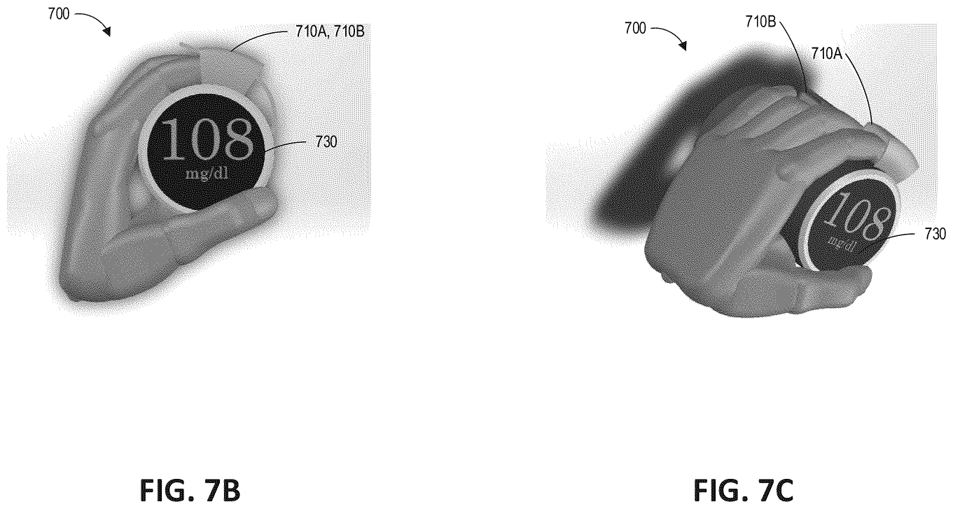

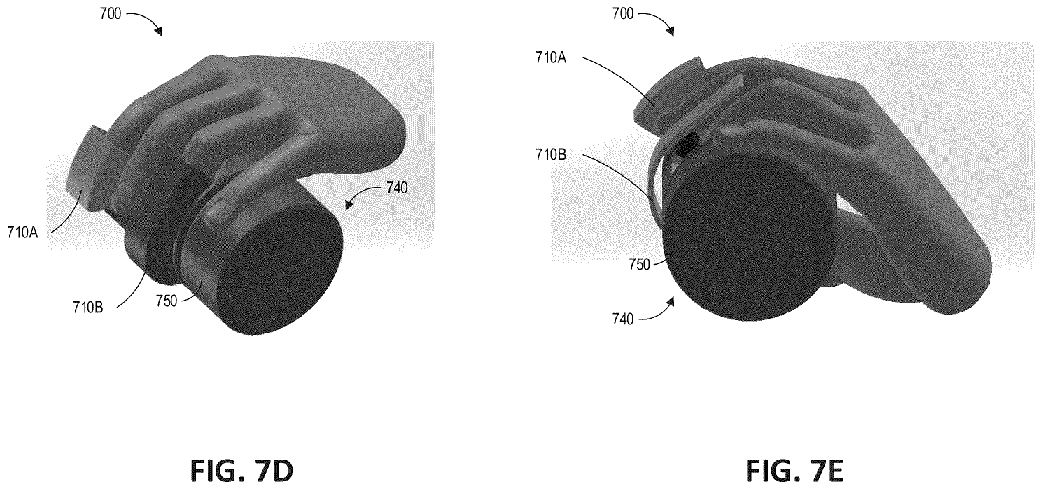

FIGS. 7A-7E show perspective views of an example multi-sensor device 700. The device 700 can include multiple different types of sensors. For example, the device 700 can include components relating to pulse-oximetry sensors and/or spectroscopic sensors capable of detecting Raman emissions. The device 700 can embody all required hardware to measure one or multiple parameters directly from the sensors. The device 700 can have one or more sensors for one or more fingers on a user's hand. For example, as illustrated in FIG. 7A, a device 700 can have two sensors 710a, 710b. The device 700 can have a first sensor in a first finger placement component 710a capable of measuring a physiological parameter from a tissue site on an index finger 720a and a second sensor in a second finger placement component 710b capable of measuring a physiological parameter from a tissue site on a ring finger 720b.

As illustrated in FIGS. 7B and 7C, the device 700 can have a display 730 capable of displaying data relating to parameters measured by the system 700. For example, as described below, the display 730 can be capable of displaying a parameter value and/or displaying a graphical representation of historical parameter values.

As illustrated in FIGS. 7D and 7E, the device 700 can include a housing 740. The housing 740 can include one or more case components 750 to hold hardware components in place within the device 700. The housing 740 can include one or more finger placement components 710a, 710b that can contain sensors (not shown). The finger placement components 710a, 710b can include finger placement components, as discussed in further detail below, that can be capable of aiding finger positioning in relation to one or more sensors (not shown) within the device 700. The finger positioning components 710a, 710b can be unique to a sensor type or the same for multiple different sensors. For example, a Raman sensor can require consistent placement of a user's fingernail in relation to the sensor. Thus, finger positioning components for a Raman sensor can be capable of holding a fingernail in place. In another example, a pulse oximetry sensor may not require as similarly consistent a placement as a Raman sensor. Thus, finger positioning components for a pulse oximetry sensor can be different from the finger positioning components for a Raman sensor.

### Separable Units

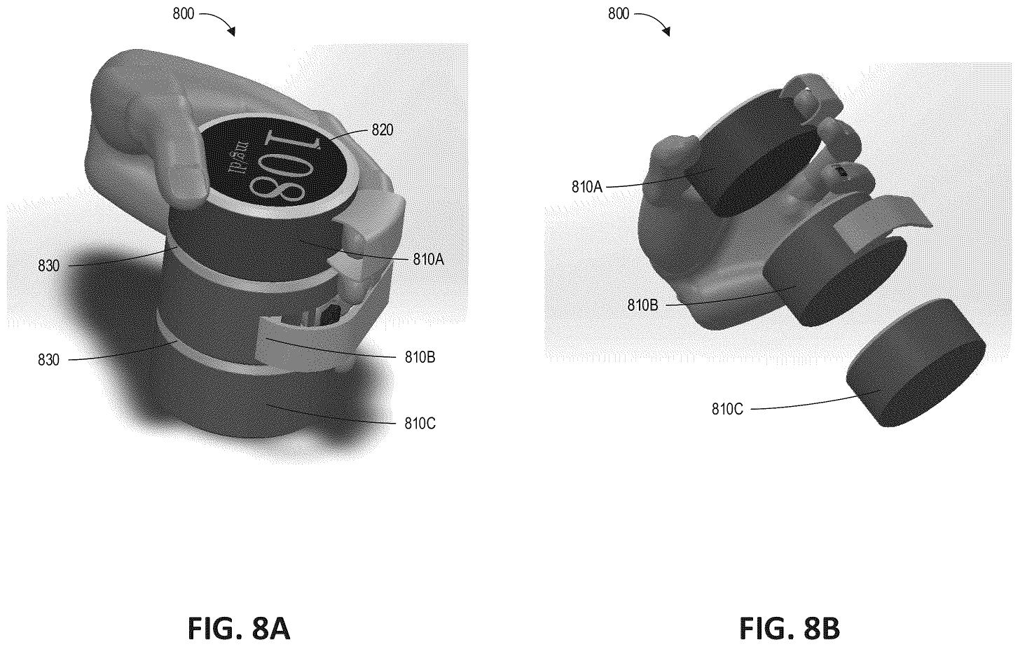

FIG. 8A shows a view of an example multi-sensor device 800 with separable units. The device 800 can include multiple units (for example, 810a, 810b, 810c) that can contain sensor and other hardware components and a display unit 820. For example, a device 800 can include a pulse oximetry unit 810a, a Raman unit 810b, and a processing unit 810c. However, more or fewer units are possible. Unit 810a can include a pulse oximetry sensor and associated hardware components. Unit 810b can include a Raman sensor and associated hardware components. Unit 810c can include other hardware components such as one or more hardware processors and power and battery components. Any unit can be a part of another unit or can be separate and the hardware components can be switched or mixed within each unit. For example, the display unit 820 can be a part of a unit 810a or a separate component. Each unit can include one or more electrical connections so as to operate the units with a single processing unit or a single power source. Additionally or alternatively, each unit can be independently operable.

The units (for example, 810a, 810b, 810c) can be separated or combined in any suitable order combination within a device 800. The units can be stackable. For example, the device can include the display unit 820 placed onto a hardware unit 810c. The hardware unit 810c can be placed onto the pulse oximetry unit 810a. The pulse oximetry unit 810a can be placed onto the Raman unit 810b. It will be understood that other combinations of units are possible.

The units (for example, 810a, 810b, 810c) can secured in place through any suitable securing mechanism. For example, units 810a, 810b, and 810c can include at least one interlocking mechanism 830, such as screw threads, clasps, notches or any other suitable interlocking mechanism. The interlocking mechanism 830 can also protect interior components of the units from outside damage, such as water damage or other sources of damage to electronic components.

### Finger Placement

As shown in FIG. 7A, a device 700 can have multiple finger placement components 710a, 710b capable of holding a sensor for measuring tissue sites on multiple fingers 720a, 720b. The finger placement components can be oriented in such a way as to allow for multiple fingers on the same hand to be simultaneously measured by the sensors in device 700. For example, the finger placement components (for example, 710a and 710b) can be oriented on the device 700 such that there is a sufficient amount of room for a user's fingers to rest comfortably. Each finger placement component can be capable of receiving a finger. A finger placement component can be capable of receiving more than one type of finger. Additionally or alternatively, in other examples, a finger placement component can be capable of receiving only one type of finger. For example, a finger placement component 710a can be capable of receive finger 720a or 720b. In another example, a finger placement component 710b can only receive finger 720b.

### Example Finger Placement Components

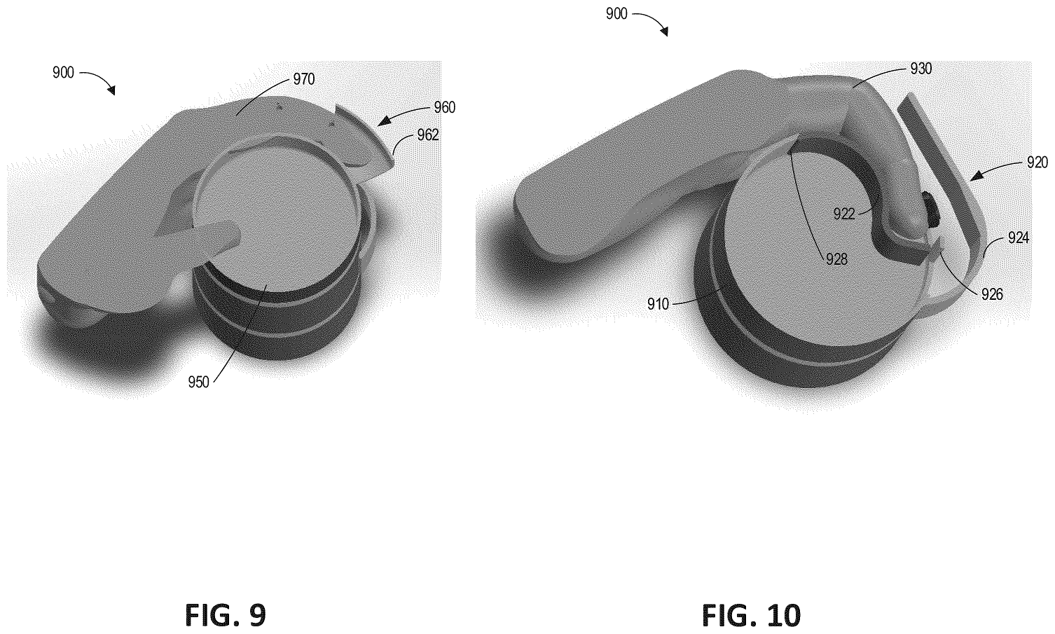

FIGS. 9 and 10 illustrate cross-sectional views of example finger placement in an example multi-sensor device 900. FIG. 9 illustrates a cross-sectional view of an example pulse-oximetry unit 950. As shown in FIG. 9, a pulse oximetry unit 950 can include a finger placement component 960. The finger placement component 960 can be composed of a single or multiple parts. For example, the finger placement component 960 can have a cover component 962. The cover component 962 can be configured to enclose or cover (partially or entirely) a tissue site. The finger placement component 920 can be of sufficient length, width, and depth to receive a human digit 970, such as an index finger, at a location close to a sensor (not shown) such that a tissue site on the human digit can be measured by the sensor.

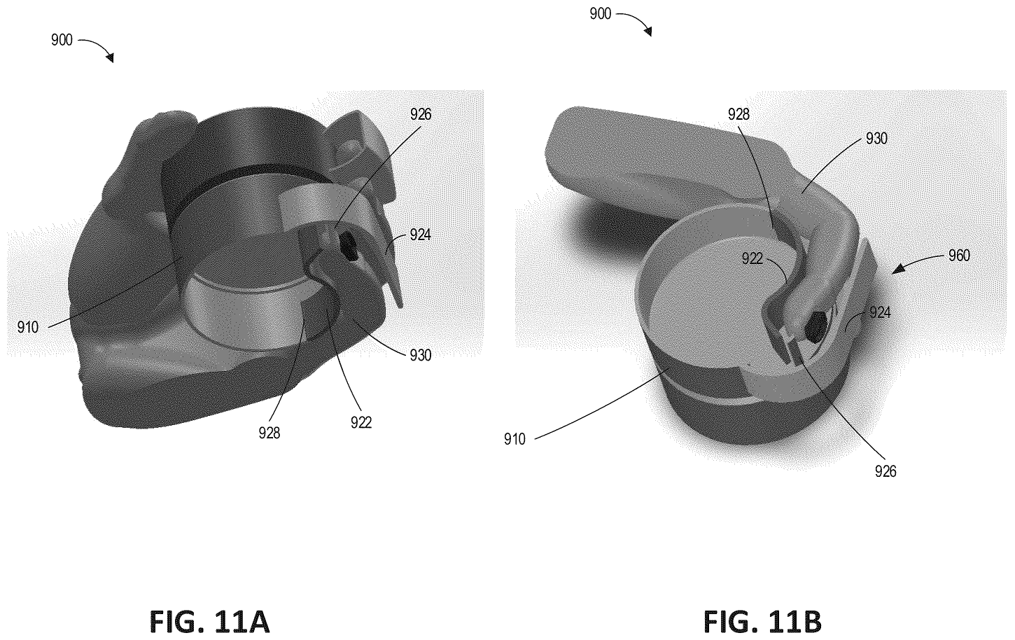

FIG. 10 illustrates a cross-sectional view and FIGS. 11A and 11B illustrate example perspective views of an example Raman sensor unit 910 with finger placement component 920. As shown in FIG. 10, a Raman sensor unit 910 can include a finger placement component 920. The finger placement component 920 can be composed of a single or multiple parts. For example, the finger placement component 920 can have a hinged component 922 designed to approximately conform to the shape of the finger 930. The hinged component 922 can be connected to a hinge 928. The hinge 928 can be part of a spring mechanism capable of pushing the finger 930 towards a sensor or a cover component 924. For example, the finger placement component 920 can additionally or alternatively have a cover component 924. The cover component 924 can house a sensor. The spring mechanism can push the finger 930 towards to sensor in the cover component 924. The cover component 924 can be configured to enclose or cover (partially or entirely) a tissue site. The finger placement component 920 can be of sufficient length, width, and depth to receive a human digit 930, such as a ring finger, at a location close to a sensor (not shown) such that a tissue site on the human digit can be measured by the sensor. The finger placement component 920 can include a finger stop 926 to prevent the finger 930 from misplacing relative to a sensor within the finger placement component 920.

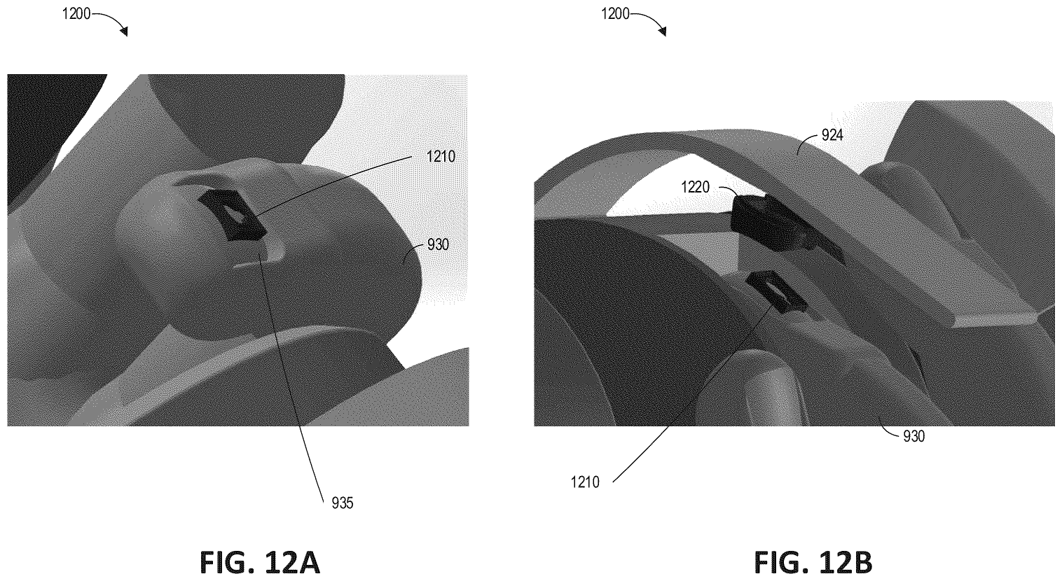

FIGS. 12A and 12B illustrate example securing components 1200 that can be part of or used in conjunction with an example Raman sensor unit 910. FIG. 12A shows an example nail guide 1210. The example nail guide 1210 can be placed onto a finger 930. For example, the nail guide 1210 can be placed onto a fingernail 935 of a finger 930. As shown in FIG. 12B, the nail guide 1210 can mate with a nail lock 1220. The nail guide 1210 can mate with the nail lock 1220 through a variety of suitable mechanisms. For example, the nail guide 1210 can mate with the nail lock 1220 with a snap-fit, a clasp, a sliding-fit or other suitable mechanism. The nail guide 1210 and the nail lock 1220 can secure the finger 930 in place at the fingernail 935 during a physiological measurement. For example, when the nail guide 1210 is in place, the nail lock 1220 can prevent the finger 930 from sliding or rotating side to side or back and forth.

The nail guide 1210 can be secured using an adhesive. The adhesive can allow the nail guide 1210 to be placed onto a finger 930 for an extended period of time. For example, the nail guide 1210 can be adhered to the nail 935 for a period of 1 day to 1 week. The benefit of adhering the nail guide 1210 for an extended period of time is that it allows for more consistent placement of the finger in the device 900 over that period. For example, a user can perform multiple non-invasive measurements of a physiological parameter over the period of a day. If the nail guide 1210 is secured in the same spot of the nail 935 for that period, then the measurements of the physiological parameter will be of approximately the same tissue site due to the finger 930 being secure in substantially the same way while the nail guide 1210 is secured in the same spot.

While the systems and methods mentioned above can be described in reference to a particular sensor or sensor unit, the components can be used for any type of sensor, sensor unit, or finger placement device or mechanism.

### Example Display Modes

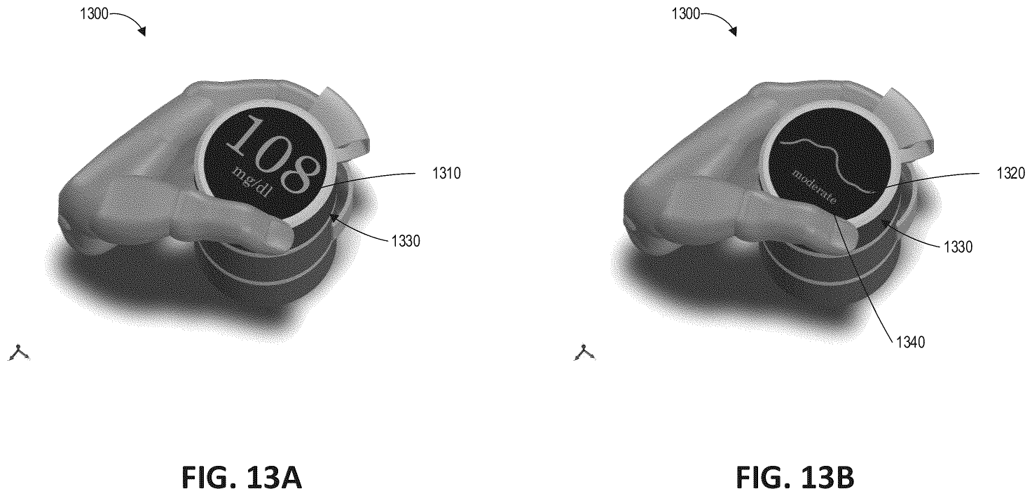

FIGS. 13A and 13B illustrate example display modes of a device 1300. As illustrated in FIG. 13A, a device 1300 can display a physiological parameter value 1310. For example, a device 1300 can be capable of measuring blood glucose. The device 1300 can have a glucometer mode, where it displays a blood glucose value on the display 1330. The units of measurement, size of text, and other relevant display settings can be customizable by the user.

As illustrated in FIG. 13B, the device 1300 can have a parameter variation display mode. For example, a user can make measurements of a physiological parameter using the device 1300 over a period of time. The device can access those values and display a graphical representation 1320 of those values on a display 1330. For example, where the physiological parameter is blood glucose, the device can display a graph of blood glucose values over time. The period of time over which a graph can display data and other relevant display settings can be customizable by the user. Additionally or alternatively, the device 1300 can display data 1340 associated with a physiological parameter measurement. For example, the device 1300 can measure blood glucose. The device 1300 can display whether the currently measurement blood glucose is within a predetermined range. For example, the device 1300 can display that the current blood glucose measurement is in a moderate range.

### Alternative Configurations

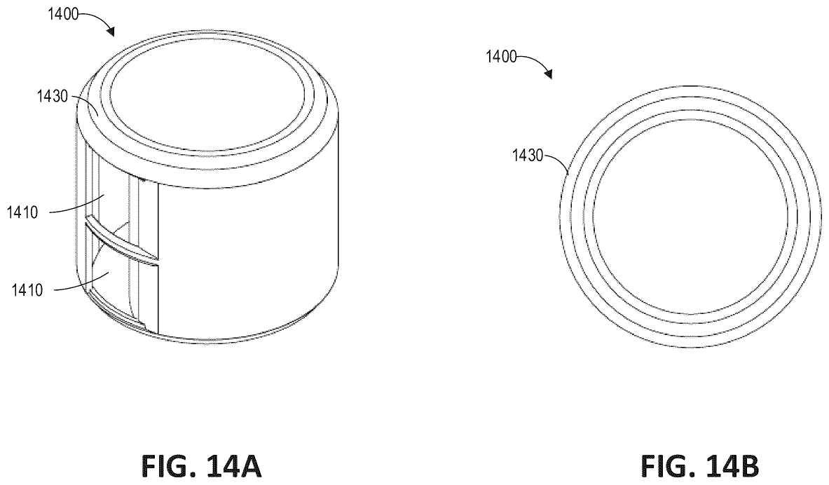

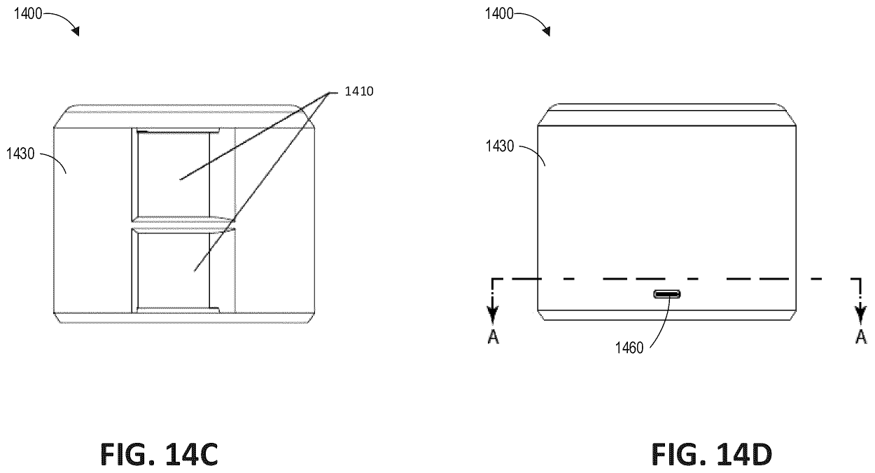

FIGS. 14A-14D show a device 1400 that can include a central housing portion 1430 with one or more finger wells 1410. The central housing portion 1430 can be an area of the device 1400 in which hardware components are stored. The central housing portion 1430 can include a single sensor or hardware unit or multiple sensor or hardware units. For example, the device 1400 can include a single unit with multiple sensors and their associated hardware. Additionally or alternatively, the device 1400 can include multiple units that can each contain one or more sensors and/or hardware components.

As illustrated in FIGS. 14A and 14C, the device 1400 can include one or more finger wells 1410. A finger well 1410 can include an opening for receiving a finger or other tissue site for measurement by a sensor within the device 1400. A finger well 1410 can be wide enough and deep enough to comfortably receive a finger of a patient. The finger well 1410 can be narrow enough so as to not allow for excessive finger movement within the device 1400. The finger well 1410 can provide a guide for a patient to insert their finger so as to guide a desired measurement site (for example, on the finger) towards a sensor 1450 within the device 1400.

As illustrated in FIG. 14D, the device 1400 can include a port 1460. The port 1460 can be capable of providing power to the device (e.g. through direct power or through battery charging), transmitting information to or from the device, or any other suitable purpose that may use an electrical connection. Additionally or alternatively, the device 1400 may wirelessly receive power or may wirelessly communicate information to or from the device.

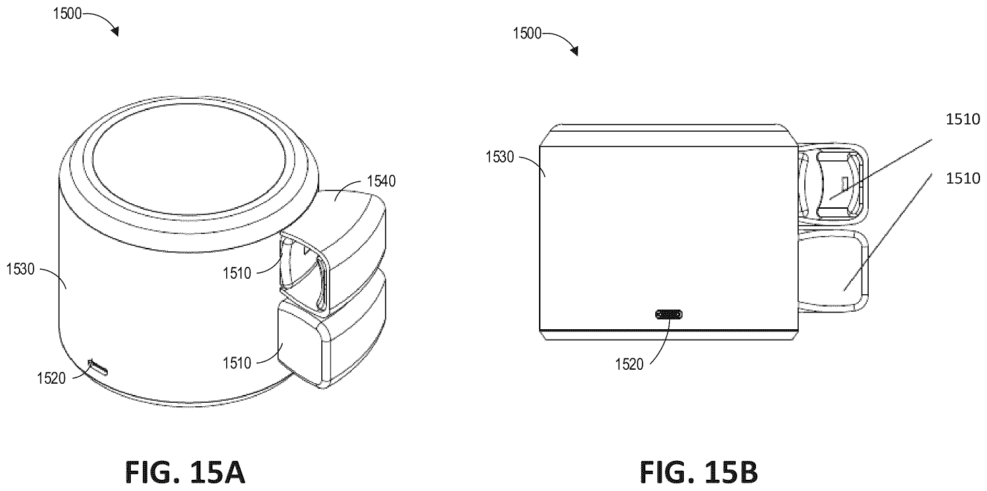

FIGS. 15A and 15B show a device 1500 that can include a central housing portion 1530 with one or more finger wells 1510. The central housing portion 1530 can be an area of the device 1400 in which hardware components are stored. The central housing portion 1530 can include a single sensor or hardware unit or multiple sensor or hardware units. For example, the device 1500 can include a single unit with multiple sensors and their associated hardware. Additionally or alternatively, the device 1500 can include multiple units that can each contain one or more sensors and/or hardware components.

The device 1500 can include one or more finger wells 1510. The finger wells can be in a finger placement component 1540 adjacent to a central housing portion 1530 of the device 1500. A finger well 1510 can include an opening for receiving a finger or other tissue site for measurement by a sensor. The sensor can be placed within the finger placement component 1540 or at another location adjacent to the finger well 1510. A finger well 1510 can be wide enough and deep enough to comfortably receive a finger of a patient. The finger well 1510 can be narrow enough so as to not allow for excessive finger movement within the device 1500. The finger well 1510 can provide a guide for a patient to insert their finger so as to guide a desired measurement site (for example, on a finger) towards a sensor (not shown) that can be part of the device 1500.

The device 1500 can include a port 1520. The port 1520 can be capable of providing power to the device (e.g. through direct power or through battery charging), transmitting information to or from the device, or any other suitable purpose that may use an electrical connection. Additionally or alternatively, the device 1500 may wirelessly receive power or may wirelessly communicate information to or from the device.

### Example Sensor Pressure Components

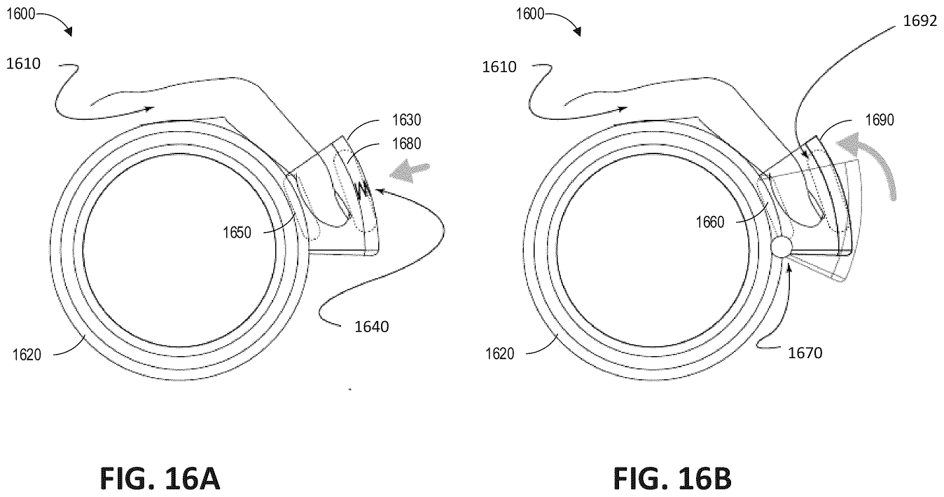

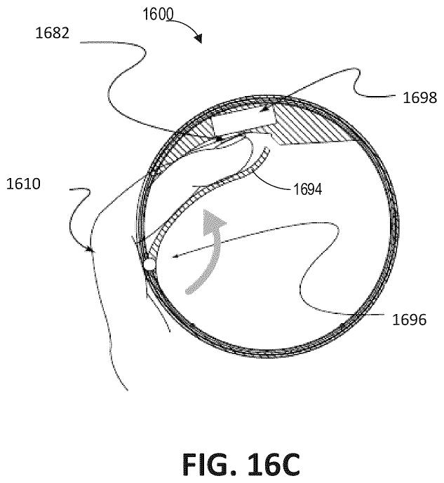

FIGS. 16A-16C show examples of sensor pressure components that can be used as part of a device 1600. A device 1600 can use one or more components to press or push the tissue site towards the sensor or the sensor towards the measurement site. For example, as illustrated in FIG. 16A, a device 1600 can include a sensor component 1680. The sensor component 1680 can include a sensor or sensor housing (not shown) and a spring system 1640. The spring system 1640 can exert a force against a sensor or sensor housing that can push the sensor towards a measurement site (for example, a portion of the finger 1610). The device 1600 may or may not include an activation component 1650. The activation component 1650 can be a pressure sensitive device, such as a button or spring. The activation component 1650 can be activated by applied pressure (for example, by a finger 1610). When activated, the activation component 1650 can cause the spring system 1640 to push the sensor towards the measurement site.

As illustrated in FIG. 16B, a device 1600 can include a sensor component 1690 and a hinge component 1670. The sensor component 1690 can include a sensor or sensor housing 1692 and may be able move with respect to a central housing component 1620. For example, the sensor housing component can operate to clamp onto a finger 1610 when pressure is exerted onto an activation component 1660. The sensor housing component can clamp onto the finger 1610 by pivoting around the hinge component 1670. The hinge component 1670 can include a spring loaded hinge. The activation component 1660 can be a pressure sensitive device, such as a button or spring. The activation component 1660 can be activated by applied pressure (for example, by a finger 1610).

As illustrated in FIG. 16C, the device 1600 can include a sensor 1650 within the device 1600. The sensor 1698 can be adjacent to the finger 1610 when placed into the device 1600 so as to measure at the tissue site. The device 1600 can have a hinged rest 1694 for the finger 1610. The hinged rest 1694 can approximately conform to the shape of the finger. The hinged rest can be connected to a spring loaded hinge 1696. When a finger 1610 is placed into the device 1600, the spring loaded hinge 1696 can push the hinged rest 1694 towards a sensor or support 1698.

With continued reference to FIG. 16C, a device 1600 can include one or more alignment structures 1682. The alignment structure 1682 can be flexible so as to approximately conform to the shape of a finger 1610 during use. For example, the alignment structure 1682 can be a silicone alignment lens. The silicone alignment lens can be capable of transmitting light or radiation from the sensor towards the finger 1610. The silicone alignment lens can be flexible so as to approximately conform to the shape of the finger 1610. The shape may be imposed while the lens or other structure is under pressure or may have a predetermined shape.

### Terminology

Many other variations than those described herein will be apparent from this disclosure. For example, depending on the embodiment, certain acts, events, or functions of any of the steps described herein can be performed in a different sequence, can be added, merged, or left out altogether (e.g., not all described acts or events are necessary for the practice of the algorithms). Moreover, in certain embodiments, acts or events can be performed concurrently. In addition, different tasks or processes can be performed by different machines and/or computing systems that can function together.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Further, the term “each,” as used herein, in addition to having its ordinary meaning, can mean any subset of a set of elements to which the term “each” is applied.

Disjunctive language such as the phrase “at least one of X, Y and Z,” unless specifically stated otherwise, is to be understood with the context as used in general to convey that an item, term, etc. may be either X, Y, or Z, or a combination thereof. Thus, such conjunctive language is not generally intended to imply that certain embodiments require at least one of X, at least one of Y and at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations.

While the above detailed description has shown, described, and pointed out novel features as applied to various embodiments, it will be understood that various omissions, substitutions, and changes in the form and details of the apparatus or method illustrated can be made without departing from the spirit of the disclosure. As will be recognized, certain embodiments of the inventions described herein can be embodied within a form that does not provide all of the features and benefits set forth herein, as some features can be used or practiced separately from others.

1.-60. (canceled)

61. A finger sensor system, the system comprising:
one or more modular components comprising:
an exterior curved wall comprising at least one finger well,
wherein the exterior curved wall is configured to allow one or more fingers of the user to rest in a natural curved position along the exterior curved wall when the cylindrical housing is held by the hand of the user,
wherein the at least one finger well comprises at least one finger sensor configured to optically measure one or more physiological parameters from a tissue site on at least one of the one or more fingers resting along the exterior curved wall.

62. The finger sensor system of claim 61 wherein the finger well is configured to be embedded in a central portion of the one or more modular components.

63. The finger sensor system of claim 61 wherein the finger well is configured to be adjacent to a central portion of the one or more modular components.

64. The finger sensor system of claim 61 wherein the finger well comprises at least one pressure component.

65. The finger sensor system of claim 64 wherein the at least one pressure component comprises a spring configured to push the at least one sensor towards an interior of the finger well.

66. The finger sensor system of claim 64 wherein the at least one pressure component comprises a clasp configured to apply pressure to a measurement site of a patient.

67. The finger sensor system of claim 61 comprising an alignment lens configured to be disposed between a finger disposed in the finger well and the at least one sensor.

68. The finger sensor system of claim 67 wherein the alignment lens comprises a flexible material.

69. The finger sensor system of claim 67 wherein the alignment lens comprises silicone.

70. The finger sensor system of claim 61, wherein at least one of the one or more modular components comprises a planar surface adjacent to the exterior curved wall.

71. The finger sensor system of claim 70, wherein the planar surface comprises a display.

72. The finger sensor system of claim 61 wherein the one or more modular components comprises a plurality of housing components configured to couple together.

73. A finger sensor system, the system comprising:
a first housing component comprising:
an exterior curved wall comprising at least one first finger well,
wherein the exterior curved wall is configured to allow one or more fingers of the user to rest in a natural curved position along the exterior curved wall when the cylindrical housing is held by the hand of the user,
wherein the at least one first finger well comprises at least one first finger sensor configured to optically measure one or more physiological parameters from a tissue site on at least one of the one or more fingers resting along the exterior curved wall, and
a second housing component configured to couple to the first housing component, the second housing component comprising at least one second finger well, the at least one second finger well comprising at least one second finger sensor.

74. The finger sensor system of claim 73, comprising a third housing component comprising one or more hardware processors.

75. The finger sensor system of claim 73 wherein the at last one first sensor comprises a spectroscopic sensor configured to measure Raman emissions.

76. The finger sensor system of claim 73 wherein the at least one first sensor comprises an optical sensor configured to measure pulse oximetry.

77. The finger sensor system of claim 73 wherein the at least one first finger well comprises a sensor cover and an internal clasp.

78. The finger sensor system of claim 77 wherein the internal clasp comprises a hinged component configured to support the first finger.

79. The finger sensor system of claim 77 wherein the sensor cover comprises the at least one first sensor.

80. The finger sensor system of claim 77 wherein the internal clasp further comprises a spring configured to push the hinged component towards the sensor cover.

81. The finger sensor system of claim 73 wherein the first housing component comprises a planar surface adjacent to the exterior curved wall and wherein the planar surface comprises a display.
